(19) United States
(12) Patent Application Publication
(10) Pub. No.: US 2019/0239857 A1
(43) Pub. Date: Aug. 8, 2019

(54) Ultrasonic Diagnostic Apparatus

(71) Applicant: Hitachi, Ltd., Chiyoda ku Tokyo (JP)

(72) Inventor: Hideaki NAKAJIMA, Chiyoda-ku, Tokyo (JP)

(21) Appl. No.: 16/335,766

(22) PCT Filed: Dec. 5, 2017
(86) PCT No.: PCT/JP2017/043576

§ 371 Date: Mar. 22, 2019

(30) Foreign Application Priority Data
Dec. 28, 2016 (JP) 2016-254635

(51) Int. Cl. A61B 8/08; A61B 8/06; A61B 8/14; A61B 8/00

(52) U.S. Cl. CPC A61B 8/5246; A61B 8/06; A61B 8/488; A61B 8/54; A61B 8/463; A61B 8/5223; A61B 8/14

(57) ABSTRACT

A scanning range for a B-mode image is divided into a plurality of scanning regions, and a division transmission and reception is performed in which transmission and reception for each scanning region and transmission and reception that targets a region of interest for a color flow are alternately repeated. A control unit determines a transmission and reception condition of the division transmission and reception based on required speed information required in a color flow image, and controls the division transmission and reception by a transmission and reception unit in accordance with the determined transmission and reception condition.

### TECHNICAL FIELD

The present invention relates to an ultrasonic diagnostic apparatus, and more particularly to an ultrasonic diagnostic apparatus that forms a color flow image.

### BACKGROUND ART

An ultrasonic diagnostic apparatus forms and displays an ultrasonic image based on a reception signal obtained by transmitting and receiving ultrasonic waves. For example, a B-mode image is well known as the ultrasonic image. Further, an apparatus is also known to display speed information obtained from a moving body such as a blood flow in a living body based on a reception signal obtained by transmitting and receiving ultrasonic waves. For example, PTL 1 and PTL 2 disclose a technique relating to a color flow image (a color Doppler image) that two-dimensionally visualizes speed information of a moving body based on Doppler information obtained from the living body by using ultrasonic waves.

### PRIOR ART LITERATURE

### Patent Literature

PTL 1: JP-A-2014-42823

PTL 2: JP-A-4-170943

### SUMMARY OF INVENTION

### Technical Problem

When obtaining a color flow image, transmission and reception for a B-mode image and transmission and reception for a color flow is performed. Then, the color flow image are formed by representing speed information on a B-mode image with a color. The speed information is based on reception information obtained by the transmission and reception for the color flow, and the B-mode image is based on reception information obtained by the transmission and reception for the B-mode image.

By using the color flow image, a user such as a doctor or a laboratory technician can diagnose a state of a moving body such as a blood flow in a living body based on the speed information on the B-mode image represented by the color. For example, a position of a portion to be diagnosed is confirmed based on a tomographic image of a tissue projected as the B-mode image, and a motion state such as the blood flow in the portion to be diagnosed is diagnosed.

In general, in diagnosis using the color flow image, the motion state such as the blood flow in the portion to be diagnosed is a diagnosis center, and the tomographic image of the tissue projected as the B-mode image is information used for grasping the position of the portion to be diagnosed and the like. That is, in the color flow image, the B-mode image projected on a background is generally auxiliary information used for diagnosing the motion state such as the blood flow.

An object of the invention is to provide an improvement technique relating to a transmission and reception control of ultrasonic waves used for obtaining a color flow image. For example, in view of the above situation, it is desirable to provide a control in which a transmission and reception for a color flow is prioritized over a transmission and reception for a B-mode image.

### Solution to Problem

An ultrasonic diagnostic apparatus suitable for satisfying the above object includes a transmission and reception unit that, upon performing transmission and reception for a B-mode image for each scanning region over a plurality of scanning regions obtained by dividing a scanning range of the B-mode image and performing transmission and reception for a color flow that targets a region of interest set within the scanning range, performs a division transmission and reception in which the transmission and reception for each scanning region and the transmission and reception that targets the region of interest are alternately repeated; an image forming unit that forms a color flow image that shows speed information on a B-mode image, the speed information being based on reception information obtained from the region of interest, and the B-mode image being based on reception information obtained from the scanning range including the plurality of scanning regions; and a control unit that determines a transmission and reception condition for the division transmission and reception based on required speed information required in the color flow image and controls the division transmission and reception by the transmission and reception unit according to the determined transmission and reception condition.

In the apparatus including the above configurations, the transmission and reception condition of the division transmission and reception is determined based on the required speed information required in the color flow image, and the division transmission and reception is controlled in accordance with the determined transmission and reception condition. For example, the division transmission and reception that includes the transmission and reception for the B-mode image is controlled so that a maximum speed required in the color flow image can be diagnosed. As described above, for example, a control in which the transmission and reception for the color flow is prioritized over the transmission and reception for the B-mode image is implemented according to the apparatus including the above configurations.

In a preferable specific example, an initial condition of the division transmission and reception is determined based on limit speed information that can be implemented in the color flow image, and the control unit determines the transmission and reception condition of the division transmission and reception based on the initial condition and the required speed information. For example, the control unit determines the transmission and reception condition of the division transmission and reception based on the required speed information while maintaining the initial condition determined based on the limit speed information.

In a preferable specific example, the initial condition of the division transmission and reception includes a division number in a case of dividing the scanning range of the B-mode image into the plurality of scanning regions and a beam number of ultrasonic beams in each scanning region, and the control unit determines the transmission and reception condition of the division transmission and reception based on the division number, the beam number, and the required speed information. For example, the control unit determines the transmission and reception condition of the division transmission and reception based on the required speed information while maintaining at least one of the division number and the beam number.

In a preferable specific example, the transmission and reception condition of the division transmission and reception includes a length of dummy period of time that is provided between the transmission and reception for each scanning region and the transmission and reception that targets the region of interest, and the control unit determines the length of the dummy period of time based on the required speed information. For example, the control unit determines the length of the dummy period of time based on the initial condition and the required speed information. For example, the control unit determines the length of the dummy period of time based on the required speed information while maintaining the initial condition. Further, the control unit may determine the length of the dummy period of time based on the division number, the beam number, and the required speed information. For example, the control unit determines the length of the dummy period of time based on the required speed information while maintaining at least one of the division number and the beam number.

### Advantageous Effect

According to the invention, an improvement technique relating to the transmission and reception control of the ultrasonic waves used for obtaining the color flow image is provided. For example, according to a preferred aspect of the invention, the control in which the transmission and reception for the color flow is prioritized over the transmission and reception for the B-mode image is provided.

### BRIEF DESCRIPTION OF DRAWINGS

FIG. 1 shows a specific example of an ultrasonic diagnostic apparatus suitable for implementing the invention.

FIG. 2 shows a specific example relating to transmission and reception of ultrasonic waves by the ultrasonic diagnostic apparatus in FIG. 1.

FIG. 3 shows a relationship between a frame rate in transmission and reception for a color flow and transmission and reception time for each scanning region.

FIG. 4 shows a specific example of an initial condition and a transmission and reception condition of a division transmission and reception.

FIG. 5 shows a display example of a color flow image obtained by the division transmission and reception.

### DESCRIPTION OF EMBODIMENTS

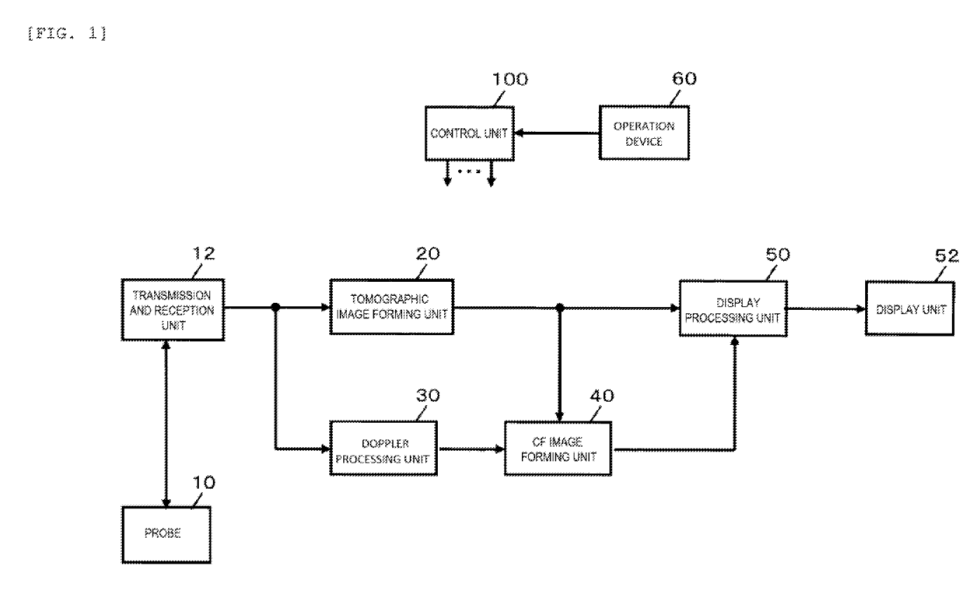

FIG. 1 shows a specific example of an ultrasonic diagnostic apparatus suitable for implementing the invention. A probe 10 is an ultrasonic probe that transmits and receives ultrasonic waves, and scans a diagnosis region that includes a diagnosis target in an object (a living body) with an ultrasonic beam. In the specific example shown in FIG. 1, the probe 10 is preferably a sector scanning probe, a convex scanning probe, and the like, and a linear scanning probe and the like may also be used.

A transmission and reception unit 12 controls transmission of a plurality of vibration elements provided in the probe 10 to form a transmission beam, and scans the diagnosis region with the transmission beam. Further, the transmission and reception unit 12 performs phasing addition processing on a plurality of reception signals obtained from the plurality of vibration elements to form a reception beam, and collects the reception signals from the whole diagnosis region. That is, the transmission and reception unit 12 has functions of a transmission beam former and a reception beam former.

A tomographic image forming unit 20 forms image data of a B-mode image (a tomographic image) of the diagnosis region based on the reception signal collected from the diagnosis region, for example, an image data of a tomographic image relating to a tissue such as a liver to be diagnosed.

A Doppler processing unit 30 obtains Doppler information from the reception signal collected from the diagnosis region. The Doppler processing unit 30 measures a Doppler shift that is generated in the reception signal of the ultrasonic waves obtained from a moving body such as a blood flow by, for example, a known Doppler processing, and obtains Doppler data in an ultrasonic beam direction (Doppler components in a beam direction) about the moving body such as the blood flow.

A color flow (CF) image forming unit 40 forms image data of a color flow image that shows speed information on the B-mode image formed in the tomographic image forming unit 20. The speed information is based on Doppler information (the Doppler data) obtained from the Doppler processing unit 30. For example, the CF image forming unit 40 forms image data of a known color flow image (a color Doppler image) in which a speed at each point in the tomographic image of the diagnosis region (for example, in the blood flow) is represented by a color and the like.

A display processing unit 50 forms a display image based on the image data of the tomographic image (the B-mode image) obtained from the tomographic image forming unit 20 and the image data of the color flow image obtained from the CF image forming unit 40. The formed display image is displayed on a display unit 52.

A control unit 100 completely controls an inside of the ultrasonic diagnostic apparatus in FIG. 1. An instruction received from a user such as a doctor or a laboratory technician via an operation device 60 is also reflected in an overall control by the control unit 100.

The transmission and reception unit 12, the tomographic image forming unit 20, the Doppler processing unit 30, the CF image forming unit 40, and the display processing unit 50 of configurations (units with reference numerals) shown in FIG. 1 may be implemented by using hardware such as an electric electronic circuit or a processor, and a device such as a memory may be used as necessary in the implementation. At least a part of functions corresponding to the units may be implemented by a computer. That is, at least a part of the functions corresponding to the units may be implemented by cooperation of hardware such as a CPU, a processor, and a memory, and software (a program) that defines an operation of the CPU and the processor.

A preferable specific example of the display unit 52 is a liquid crystal display, an organic electroluminescence (EL) display, and the like, and the operation device 60 can be implemented by at least one of a mouse, a keyboard, a trackball, a touch panel, other switches, and the like. Then, the control unit 100 can be implemented, for example, by cooperation of hardware such as a CPU, a processor, and a memory, and software (a program) that defines an operation of the CPU and the processor.

All configurations of the ultrasonic diagnostic apparatus in FIG. 1 are described as above. Next, processing and functions that are implemented by the ultrasonic diagnostic apparatus in FIG. 1 will be described in detail. In the following description of configurations (a part) shown in FIG. 1, the reference numerals in FIG. 1 are used.

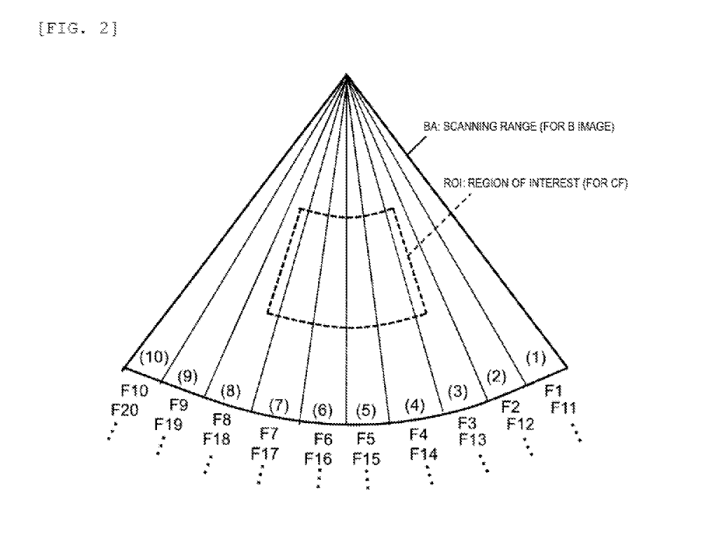

FIG. 2 shows a specific example relating to transmission and reception of ultrasonic waves by the ultrasonic diagnostic apparatus in FIG. 1. FIG. 2 shows a specific example of a scanning range BA for the B-mode image and a region of interest ROI for the CF (the color flow). In the specific example shown in FIG. 2, the scanning range BA for the B-mode image is a fan-shaped region surrounded by a solid line, and the region of interest ROI for the CF is set within the scanning range BA. Note that the entire scanning range BA may be the region of interest ROI.

The transmission and reception unit 12 scans scanning range BA with an ultrasonic beam (a transmission beam and a reception beam) to perform transmission and reception for the B-mode image. The tomographic image forming unit 20 forms the image data of the B-mode image (the tomographic image) based on reception information (luminance information of an echo) obtained from the scanning range BA by the transmission and reception.

The transmission and reception unit 12 scans the region of interest ROI with an ultrasonic beam (the transmission beam and the reception beam) to perform the transmission and reception for the color flow. The Doppler processing unit 30 obtains the speed information of the moving body such as the blood flow based on reception information (Doppler shift information) obtained from the region of interest ROI by the transmission and reception.

Then, the CF image forming unit 40 forms the image data of the color flow image that shows the speed information in the region of interest ROI on the B-mode image that corresponds to the scanning range BA.

In a color flow mode in which the image data of the color flow image is formed, the transmission and reception unit 12 performs a division transmission and reception described below. In the division transmission and reception, the scanning range BA of the B-mode image is divided into a plurality of scanning regions. The transmission and reception unit 12 performs transmission and reception for the B-mode image for each scanning region over the plurality of scanning regions.

For example, as shown in the specific example shown in FIG. 2, the scanning range BA is divided into a plurality of scanning regions (1) to (10). If the number of transmission beams that scan over the entire scanning range BA is 100, the scanning range BA is divided into the plurality of scanning regions (1) to (10) so that each of the scanning regions (1) to (10) includes ten transmission beams.

Meanwhile, the region of interest ROI in the CF mode is not divided. In formation of the color flow image, formation of the ultrasonic beam is repeated for a plurality of times for each beam address of a plurality of beam addresses that corresponds to a plurality of beams that pass through the region of interest ROI, and the reception signal is collected for a plurality of times for each beam address. In collection of the reception signal, a known high frame rate method described in, for example, PTL 1 (JP-A-2014-42823) is used.

In the high frame rate method, for example, in the transmission and reception for the color flow over a plurality of frames that target the region of interest ROI, transmission and reception is performed once at each beam address for each frame. Then, the formation of the ultrasonic beam is repeated for a plurality of frames (a plurality of times) for each beam address of the plurality of beam addresses that corresponds to the plurality of beams that pass through the region of interest ROI, and the reception signal is collected for a plurality of frames (a plurality of times) for each beam address. In this manner, the speed information (the Doppler information) is derived from the reception information obtained for a plurality of frames (a plurality of times) for each position (a position specified by the beam address and the depth) in the region of interest ROI.

Therefore, in the high frame rate method, a frame rate (a frame frequency) in the transmission and reception for the color flow is a pulse repetition frequency (PRF) in Doppler measurement. There is a relationship of Fd=PRF/2 between the pulse repetition frequency (PRF) in the Doppler measurement and a maximum Doppler shift frequency (Fd) that can be detected without an aliasing effect. That is, a range of the Doppler frequency that can be detected without the aliasing effect is ±PRF/2.

The transmission and reception unit 12 performs the division transmission and reception using the high frame rate method. That is, the transmission and reception unit 12 performs the division transmission and reception in which the transmission and reception for each scanning region forming the scanning range BA for the B-mode image and the transmission and reception for the color flow that targets the region of interest ROI are alternately repeated.

For example, in the specific example shown in FIG. 2, after transmission and reception for the color flow that targets a first frame (F1) of the region of interest ROI is performed, transmission and reception for the B-mode image that targets a scanning region (1) is performed. Subsequently, after transmission and reception for the color flow that targets a second frame (F2) of the region of interest ROI is performed, transmission and reception for the B-mode image that targets a scanning region (2) is performed. Further, after transmission and reception for the color flow that targets a third frame (F3) of the region of interest ROI is performed, transmission and reception for the B-mode image that targets a scanning region (3) is performed. The transmission and reception that targets the region of interest ROI and the transmission and reception that targets each scanning region are alternately repeated in a fourth frame (F4) and subsequent frames.

In this manner, when the transmission and reception for the color flow that targets the first frame (F1) to a tenth frame (F10) of the region of interest ROI and the transmission and reception for the B-mode image that targets the scanning regions (1) to (10) are performed and the reception signal forming the entire scanning range BA for the B-mode image is obtained, the image data of the B-mode image that corresponds to the scanning range BA for the B-mode image is formed by the tomographic image forming unit 20. Further, the image data of the color flow image that shows the speed information in the region of interest ROI is formed on the B-mode image that corresponds to the scanning range BA by the CF image forming unit 40.

Thereafter, the division transmission and reception is performed. For example, in the specific example shown in FIG. 2, when the transmission and reception for the color flow that targets the tenth frame (F10) of the region of interest ROI and the transmission and reception for the B-mode image that targets the scanning region (10) are performed, and after the transmission and reception for the color flow that targets an eleventh frame (F11) of the region of interest ROI is performed, the transmission and reception for the B-mode image that targets the scanning region (1) is performed. Subsequently, after the transmission and reception for the color flow that targets a twelfth frame (F12) of the region of interest ROI is performed, the transmission and reception for the B-mode image that targets a scanning region (2) is performed. Thus, the transmission and reception that targets the region of interest ROI and the transmission and reception that targets each scanning region are alternately repeated in a thirteenth frame (F13) and subsequent frames.

The B-mode image in each scanning region in which the transmission and reception is newly performed is partially updated in the eleventh frame (F11) and subsequent frames. For example, the image data of the B-mode image that corresponds to the scanning range BA is formed based on the reception signal of the scanning region (1) updated in the eleventh frame (F11) and the reception signal from the scanning region (2) to the scanning region (10) obtained from the second frame (F2) to the tenth frame (F10). The B-mode image in each scanning region in which the transmission and reception is newly performed is partially updated in the twelfth frame (F12) and subsequent frames.

When the reception signal of the scanning region is updated, it is desirable to perform a smoothing processing and the like on the reception signal between the updated scanning region and a scanning region adjacent thereto. For example, when the reception signal of the scanning region (1) in the eleventh frame (F11) is updated, a relatively large time difference occurs between the reception signal of the scanning region (1) and the reception signal of the scanning region (2) that is already obtained in the second frame (F2). Therefore, it is desirable to perform the smoothing processing and the like on the reception signal between the scanning region (1) and the scanning region (2) (in particular, near a boundary).

As described above, the ultrasonic diagnostic apparatus in FIG. 1 performs the division transmission and reception using the high frame rate method. In the high frame rate method, the frame rate (the frame frequency) in the transmission and reception for the color flow is the pulse repetition frequency (PRF) in the Doppler measurement. There is the relationship of Fd=PRF/2 between the pulse repetition frequency (PRF) in the Doppler measurement and the maximum Doppler shift frequency (Fd) that can be detected without the aliasing effect. That is, the range of the Doppler frequency that can be detected without the aliasing effect is ±PRF/2.

Therefore, the control unit 100 determines the pulse repetition frequency (PRF) in the Doppler measurement, that is, the frame rate FR (the frame frequency) in the transmission and reception for the color flow, so as to be able to detect a required maximum speed (the required maximum speed that is set by the user according to, for example, a diagnosis target and a diagnosis application) which is speed information required in the color flow image without the aliasing effect. The range of Doppler frequency that can be detected without the aliasing effect is ±PRF/2. When the Doppler shift frequency that corresponds to the required maximum speed is Fdd, the control unit 100 sets PRF=2×Fdd and sets the frame rate FR in the transmission and reception for the color flow as FR=PRF=2×Fdd.

Further, the control unit 100 determines the transmission and reception condition of the division transmission and reception such that the frame rate FR in the transmission and reception for the color flow is FR=2×Fdd. In the division transmission and reception in which the transmission and reception for each scanning region and the transmission and reception that targets the region of interest ROI are alternately repeated, the frame rate of the transmission and reception that targets the region of interest ROI for the color flow is changed according to transmission and reception time for each scanning region. Therefore, when the Doppler shift frequency that corresponds to the required maximum speed is Fdd, the control unit 100 determines the transmission and reception time for each scanning region such that the frame rate FR in the transmission and reception for the color flow is FR=2×Fdd.

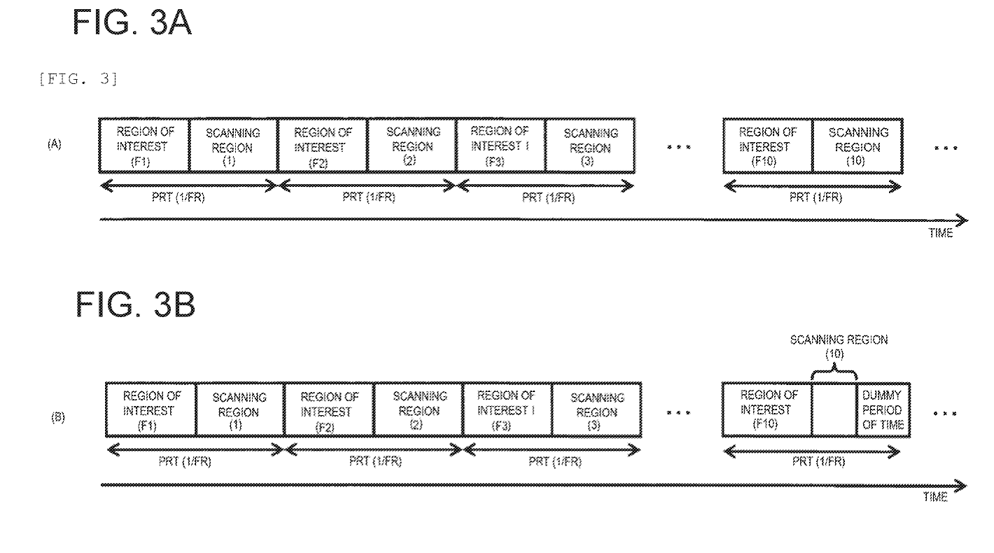

FIG. 3 shows a relationship between the frame rate in the transmission and reception for the color flow and the transmission and reception time for each scanning region. In FIG. 3, a specific example of a transmission and reception sequence that corresponds to the division transmission and reception described with reference to FIG. 2 is shown.

As shown in FIG. 3A, in the division transmission and reception, the transmission and reception for each scanning region and the transmission and reception that targets the region of interest ROI are alternately repeated. That is, after the transmission and reception for the color flow that targets the first frame (F1) of the region of interest ROI is performed, the transmission and reception for the B-mode image that targets the scanning region (1) is performed, and subsequently, the transmission and reception for the B-mode image that targets the second frame (F2) of the region of interest ROI is performed. The transmission and reception for each scanning region and the transmission and reception that targets the region of interest ROI are alternately repeated in the third frame (F3) and subsequent frames.

When the Doppler shift frequency that corresponds to the required maximum speed is Fdd, the control unit 100 determines the transmission and reception time for each scanning region such that the frame rate FR in the transmission and reception for the color flow is FR=2×Fdd. For example, the transmission and reception time for each scanning region is adjusted so that a pulse repetition time PRT, which is total time of the transmission and reception time for each frame of the region of interest ROI and the transmission and reception time for each scanning region, is PRT=1/FR (FR=2×Fdd).

The transmission and reception time for each frame of the region of interest ROI varies depending on a magnitude of the region of interest ROI (the beam number), a depth (a lower limit position of the region of interest ROI), and the like. In the color flow mode, for example, the magnitude of the region of interest ROI, the depth, and the like are set by the user such as a doctor or a laboratory technician according to a diagnosis target, a diagnostic application, and the like. Therefore, it is desirable to set the pulse repetition time PRT by only adjusting the transmission and reception time for each scanning region, while fixing the transmission and reception time for each frame of the region of interest ROI by preferentially maintaining these settings by the user, for example.

In addition, if a total beam number in the entire scanning range BA for the B-mode image and a display depth of each beam (the transmission and reception time for each beam) are known, the beam number for each scanning region may be determined based on the transmission and reception time for each scanning region. Further, the region number (the division number) of a plurality of scanning regions may be determined based on the beam number for each scanning region and the total beam number in the entire scanning range BA.

In addition, it is desirable that the pulse repetition time PRT is constant over a plurality of frames so that the range of the Doppler frequency±PRF/2, which can be detected without the aliasing effect, does not vary. Therefore, when the total time of the transmission and reception time for each frame of the region of interest ROI and the transmission and reception time for each scanning region is not constant, it is desirable that a dummy period of time is provided between the transmission and reception for each frame of the region of interest ROI and the transmission and reception for each scanning region, and the dummy period of time is adjusted so that a time length of a sum of the transmission and reception time for each frame of the region of interest ROI, the transmission and reception time for each scanning region, and the dummy period of time is constant over the plurality of scanning regions.

FIG. 3B shows a specific example of the dummy period of time. In the specific example shown in FIG. 3B, the dummy period of time is provided immediately after the transmission and reception for the color flow that targets the tenth frame (F10) of the region of interest ROI and the transmission and reception for the B-mode image that targets the scanning region (10) are performed. Note that the dummy period of time may be provided between the transmission and reception for the color flow that targets the tenth frame (F10) of the region of interest ROI and the transmission and reception for the B-mode image that targets the scanning region (10).

For example, when a division is performed with the same beam number for each of the scanning regions (1) to (9) and only the scanning region (10) has a smaller beam number than the other scanning regions, as shown in the specific example shown in FIG. 3B, the dummy period of time is provided immediately after (or immediately before) the transmission and reception for the B-mode image that targets the scanning region (10) is performed, and the dummy period of time is adjusted so that the total time of the transmission and reception time for each frame of the region of interest ROI, the transmission and reception time, and the dummy period of time for each scanning region is constant over a plurality of scanning regions. Accordingly, it is possible to form the color flow image so that the range of the Doppler frequency±PRF/2 does not vary, that is, the maximum speed that can be detected without the aliasing effect does not change. The Doppler frequency can be detected without the aliasing effect.

Further, it is preferable that the control unit 100 determines the initial condition of the division transmission and reception based on the limit speed information that can be implemented in the formation of the color flow image by the ultrasonic diagnostic apparatus in FIG. 1. For example, the control unit 100 determines the pulse repetition frequency (PRF) in the Doppler measurement, that is, the frame rate FR (the frame frequency) in the transmission and reception for the color flow, so that the limit maximum speed, which is a specific example of the limit speed information that can be realized in the color flow image, can be detected without the aliasing effect. The range of Doppler frequency that can be detected without the aliasing effect is ±PRF/2. Therefore, when the Doppler shift frequency that corresponds to the limit maximum speed is Fdₘₐₓ, the control unit 100 sets PRF=2×Fdₘₐₓ, sets the frame rate FR in the transmission and reception for the color flow as FR=PRF=2×Fdₘₐₓ, and determines the initial condition of the division transmission and reception.

Since the limit maximum speed is the maximum speed that can be implemented by the ultrasound diagnostic apparatus in FIG. 1, the maximum speed required in the color flow image is limited to the limit maximum speed or less. The control unit 100 determines the transmission and reception condition of the division transmission and reception according to the required maximum speed while maintaining the initial condition determined based on the limit maximum speed.

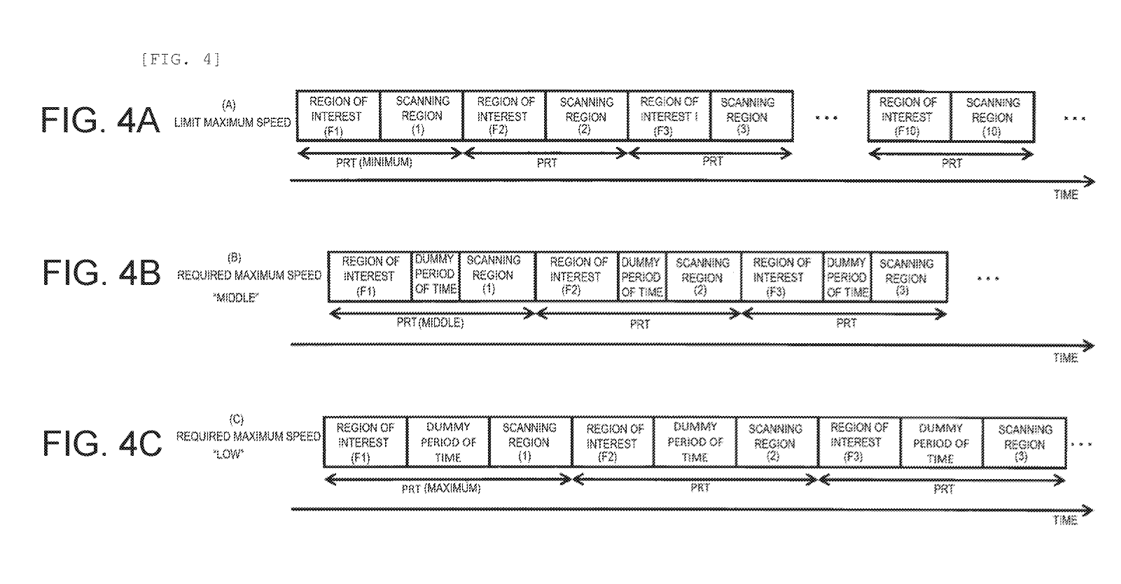

FIG. 4 shows a specific example of the initial condition and the transmission and reception condition of the division transmission and reception. FIG. 4 shows a transmission and reception sequence that corresponds to the division transmission and reception described with reference to FIG. 2.

FIG. 4A shows a transmission and reception sequence that corresponds to the limit maximum speed. As shown in FIG. 4A, in the division transmission and reception, the transmission and reception for each scanning region and the transmission and reception that targets the region of interest ROI are alternately repeated. That is, the transmission and reception for the color flow that targets the first frame (F1) of the region of interest ROI and the transmission and reception for the B-mode image that targets the scanning region (1) are performed. Subsequently, the transmission and reception for the color flow that targets the second frame (F2) of the region of interest ROI and the transmission and reception for the B-mode image that targets the scanning region (2) are performed. The transmission and reception for each scanning region and the transmission and reception that targets the region of interest ROI are alternately repeated in the third frame (F3) and subsequent frames.

When the Doppler shift frequency that corresponds to the limit maximum speed is Fdₘₐₓ, the control unit 100 determines the initial condition of the division transmission and reception such that the frame rate FR in the transmission and reception for the color flow is FR=2×Fdₘₐₓ. For example, the initial condition of the division transmission and reception is determined such that the pulse repetition time PRT, which is the total time of the transmission and reception time for each frame of the region of interest ROI and the transmission and reception time for each scanning region, is PRT=1/FR (FR=2×Fdₘₐₓ).

The transmission and reception time for each frame of the region of interest ROI varies depending on the magnitude (the beam number) of the region of interest ROI, the depth (the lower limit position of the region of interest ROI), and the like. In the color flow mode, for example, the magnitude of the region of interest ROI, the depth, and the like are set by the user such as a doctor or a laboratory technician according to a diagnosis target, a diagnostic application, and the like. Therefore, it is desirable that the setting relating to the region of interest ROI specified by the user is maintained as the initial condition. Therefore, the control unit 100, by adjusting the condition that relates to the division of the scanning range BA for the B-mode image, for example, the division number in the case of dividing the scanning range BA for the B-mode image into the plurality of scanning regions and the beam number of the ultrasonic beams in each scanning region, adjusts the transmission and reception time for each scanning region so as to set the initial condition such that the pulse repetition time is PRT=1/FR (FR=2×Fdₘₐₓ).

Thus, when the initial condition is determined based on the limit maximum speed, the control unit 100 determines the transmission and reception condition of the division transmission and reception according to the required maximum speed while maintaining the initial condition determined based on the limit maximum speed.

FIGS. 4B and 4C show a specific example of determining the transmission and reception condition of the division transmission and reception based on the required maximum speed while maintaining the initial condition in FIG. 4A. The required maximum speed is limited to the limit maximum speed or less.

For example, the control unit 100 determines the transmission and reception condition according to the required maximum speed while maintaining the initial condition (for example, at least one of the division number of the plurality of scanning regions and the beam number of the ultrasonic beams in each scanning region) determined based on the limit maximum speed.

For example, the dummy period of time is provided between the transmission and reception for each scanning region and the transmission and reception that targets the region of interest, and the control unit 100 adjusts the length of the dummy period of time according to the required maximum speed while maintaining the setting relating to the region of interest ROI determined in FIG. 4A and the setting relating to the beam number of each scanning region.

For example, as shown in FIG. 4B, when the Doppler shift frequency that corresponds to the required maximum speed is Fdd, the control unit 100 determines the length of the dummy period of time such that the frame rate FR in the transmission and reception for the color flow is FR=2×Fdd. For example, the same condition as that of the transmission and reception in FIG. 4A is applied to the transmission and reception for each frame of the region of interest ROI and the transmission and reception for each scanning region, that is, the transmission and reception time for each frame of the region of interest ROI and the transmission and reception time for each scanning region are maintained, and the length of the dummy period of time is adjusted such that the pulse repetition time PRT, which is the total time of the transmission and reception time for each frame of the region of interest ROI, the dummy period of time, and the transmission and reception time for each scanning region, is PRT=1/FR (FR=2×Fdd).

FIG. 4C shows a specific example in a case where the required maximum speed is lower (smaller) than that in FIG. 4B. Also in the specific example shown in FIG. 4C, the control unit 100 adjusts the length of the dummy period of time according to the required maximum speed while maintaining the setting relating to the region of interest ROI determined in FIG. 4A and the setting relating to the plurality of scanning regions (including the division number and the beam number for each scanning region). Since the required maximum speed is lower than that in FIG. 4B, the dummy period of time in FIG. 4C is longer than that in FIG. 4B.

In the specific example described with reference to FIG. 4, the division transmission and reception can be implemented at a frame rate that corresponds to the required maximum speed by adjusting the length of the dummy period of time, while maintaining the setting relating to the region of interest ROI determined as the initial condition and the setting relating to the plurality of scanning regions (including the division number and the beam number of each scanning region). Accordingly, for example, even when the required maximum speed is changed, the color flow image can be formed while maintaining the setting relating to the region of interest ROI and the setting relating to the plurality of scanning regions, and a change in the image quality of the color flow image accompanying a change in the required maximum speed is prevented.

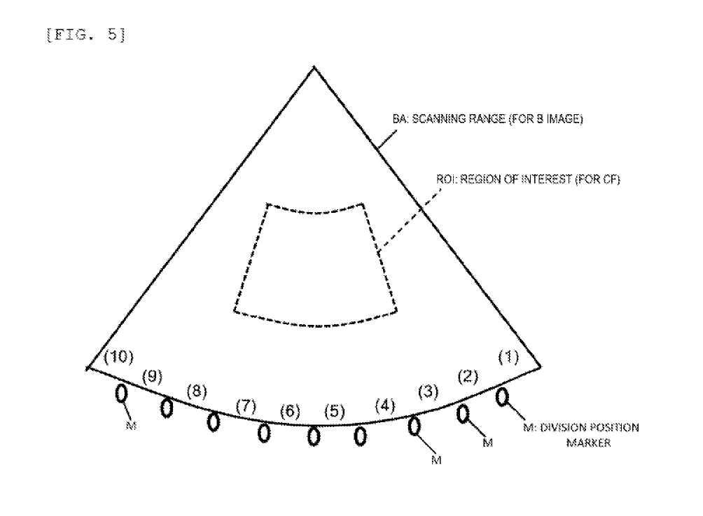

FIG. 5 shows a display example of the color flow image obtained by the division transmission and reception in FIG. 2. In the division transmission and reception shown in FIG. 2, the scanning range BA for the B-mode image is divided into the plurality of scanning regions (1) to (10). Therefore, for example, as shown in a specific example shown in FIG. 5, a display processing unit 50 may form a display image of the color flow image with a division position marker M that indicates a division position of the scanning range BA, that is, a boundary position between adjacent scanning regions (see FIG. 2). It is desirable that the division position marker M can be switched between display and non-display, for example, according to an instruction from the user.

Although the preferred embodiment of the invention has been described above, the above-described embodiment is merely an example in all respects, and does not limit the scope of the invention. The invention includes various modifications without departing from the spirit thereof.

### REFERENCE SIGN LIST

10 probe, 12 transmission and reception unit, 20 tomographic image forming unit, 30 Doppler processing unit, 40 CF image forming unit, 50 display processing unit, 52 display unit, 60 operation device, 100 control unit.

1. An ultrasonic diagnostic apparatus comprising:
a transmission and reception unit that, upon performing transmission and reception for a B-mode image for each scanning region over a plurality of scanning regions obtained by dividing a scanning range of the B-mode image and performing transmission and reception for a color flow that targets a region of interest set within the scanning range, performs a division transmission and reception in which the transmission and reception for each scanning region and the transmission and reception that targets the region of interest are alternately repeated;
an image forming unit that forms a color flow image that shows speed information on a B-mode image, the speed information being based on reception information obtained from the region of interest, and the B-mode image being based on reception information obtained from the scanning range including the plurality of scanning regions; and
a control unit that determines a transmission and reception condition for the division transmission and reception based on required speed information required in the color flow image and controls the division transmission and reception by the transmission and reception unit according to the determined transmission and reception condition.

2. The ultrasonic diagnostic apparatus according to claim 1, wherein
an initial condition of the division transmission and reception is determined based on limit speed information that is implemented in the color flow image, and
the control unit determines the transmission and reception condition of the division transmission and reception based on the initial condition and the required speed information.

3. The ultrasonic diagnostic apparatus according to claim 2, wherein
the control unit determines the transmission and reception condition of the division transmission and reception based on the required speed information while maintaining the initial condition determined based on the limit speed information.

4. The ultrasonic diagnostic apparatus according to claim 2, wherein
the initial condition of the division transmission and reception includes a division number in a case of dividing the scanning range for the B-mode image into a plurality of scan regions, and a beam number of ultrasonic beams in each scanning region, and
the control unit determines the transmission and reception condition of the division transmission and reception based on the division number, the beam number, and the required speed information.

5. The ultrasonic diagnostic apparatus according to claim 4, wherein
the control unit determines the transmission and reception condition of the division transmission and reception based on the required speed information while maintaining at least one of the division number and the beam number.

6. The ultrasonic diagnostic apparatus according to claim 1, wherein
the transmission and reception condition of the division transmission and reception includes a length of a dummy period of time provided between the transmission and reception for each scanning region and the transmission and reception that targets the region of interest, and
the control unit determines the length of the dummy period of time based on the required speed information.

7. The ultrasonic diagnostic apparatus according to claim 2, wherein
the transmission and reception condition of the division transmission and reception include a length of a dummy period of time provided between the transmission and reception for each scanning region and the transmission and reception that targets the region of interest, and
the control unit determines the length of the dummy period of time based on the initial condition and the required speed information.

8. The ultrasonic diagnostic apparatus according to claim 7, wherein
the control unit determines the length of the dummy period of time based on the required speed information while maintaining the initial condition.

9. The ultrasonic diagnostic apparatus according to claim 4, wherein
the transmission and reception condition of the division transmission and reception includes a length of a dummy period of time provided between the transmission and reception for each scanning region and the transmission and reception that targets the region of interest, and
the control unit determines the length of the dummy period of time based on the division number, the beam number, and the required speed information.

10. The ultrasonic diagnostic apparatus according to claim 9, wherein
the control unit determines the length of the dummy period of time based on the required speed information while maintaining at least one of the division number and the beam number.
